(19) United States
(12) Patent Application Publication
(10) Pub. No.: US 2005/0119574 A1
(43) Pub. Date: Jun. 2, 2005

(54) Ultrasonic imaging software and hardware pack

(75) Inventors: Charles Maerfeld, Golfe Juan (FR); Sixte de Fraguier, Valbonne (FR); Tore Sorensen, Hasselvila (NO)

Correspondence Address: LOWE HAUPTMAN GILMAN & BERNER, LLP, 1700 DIAGNOSTIC ROAD, SUITE 300, ALEXANDRIA, VA, 22314, US

(73) Assignee: THALES UL TRASONICS SAS, Neuilly Sur Seine (FR)

(21) Appl. No.: 10/506,870

(22) PCT Filed: Mar. 7, 2003
(86) PCT No.: PCT/FR03/00742

(30) Foreign Application Priority Data
Mar. 13, 2002 (FR) 02 03122

(51) Int. Cl. A61B 8/14

(52) U.S. Cl. 600/459

(57) ABSTRACT

Ultrasound imaging pack composed of a hardware part for ultrasound imaging preprocessing and of a software part intended to be installed on a microcomputer, the hardware part including at least one ultrasound probe connected to a module of electronic circuits at least part of which is configurable, this module comprising analog FEC circuits, an assembly of analog/digital converters, an array of logic electronic gates and a high throughput link between this module and the microcomputer, the software part being stored on a removable medium.

The present invention pertains to an ultrasound imaging hardware and software pack.

Since its introduction, before the end of the 1970s, ultrasound imaging has evolved a great deal. However, the general architecture of electronic imaging hardware has remained almost unchanged since electronic-scanning probes began to be used toward the end of the 1970s, this hardware assembly being dubbed an ultrasound imaging apparatus.

An ultrasound imaging apparatus is a complete apparatus which operates in an autonomous manner, with its own power supply block. Certain echographs are bulky and mounted on large casters, others, that are less bulky, are portable and furnished with handles for this purpose.

In general, top of the range apparatus are bulky and/or very expensive, since, in order to be able to produce good quality images in real time, they must include probes with a large number of sensors (preferably at least 64 sensors), and consequently a large number of circuits for processing the signals from these sensors (amplification circuits for each of the channels, circuits for separating the transmit signals from the receive signals, channel forming circuits—also referred to as beam forming circuits—, black and white imaging processors, Doppler imaging processors, image converters—called “scan converters”, etc.).

Even about fifteen years ago, out of the aforementioned circuits, only the image converter was digital. Since then, by virtue of the miniaturization of digital integrated circuits and the increase in the density of their functions, the various functions of echographs have been implemented with the aid of digital processors. Given that the echoes gathered by the probes of echographs are analog signals, the latter have to be converted into digital signals to be able to be processed by said digital processors. Echoes from each of the channels are digitized by an analog/digital converter. The number and the position in the processing chain of the signals from these converters depend in particular on the computational power of the digital processors of this processing chain, as well as on the characteristics of these converters.

The echoes are processed (amplified by low noise amplifiers with an amplification level that varies with time so as to compensate for propagation losses) in “front-end” circuits (so-called FEC, that is to say “Front End Conditioning”) which include a circuit for separating the transmit signals, which are of high level, from the receive signals, which have a low level. These FEC circuits are generally followed by analog/digital converters, themselves followed by a beam former. It should be noted that at the input of the beam former, the data stream is of the order of 20 Gbits/s for 64 channels of signals to be processed. This processing can be performed only with specialized processors designed for the purpose. Rather than implementing an analog-channel former with delay lines (which are not stable and have a limited delay), recent top of the range echographs call upon digital processors that are expensive and occupy a great deal of space. The cost of echographs and the space that they occupy can be reduced if the number of channels of the sensor is reduced, but doing so is detrimental to the quality of the echographic image.

Another recent upgrade to the production of echographs consists in using personal computers (PCs) by incorporating circuits and software specific to echographs into them. Thus, patent U.S. Pat. No. 6,325,759 discloses, for example, an ultrasound imaging apparatus whose beam former is configurable and mounted on the motherboard of a PC. Such a solution, although reducing the cost of an ultrasound imaging apparatus, is not upgradable.

U.S. Pat. No. 5,957,846 discloses a compact ultrasound imaging apparatus in which the FEC circuits and the beam former are incorporated into the housing of the probe, and are connected by a link, not described, to a portable microcomputer. For this purpose, the known device calls upon special components, CCDs, that make it possible to produce delays in analog form, in a much more efficacious manner than allowed by traditional LC circuits. Only a single analog/digital converter is necessary. This solution, although making it possible to obtain a particularly compact apparatus, exhibits all the drawbacks of analog channel forming devices, especially as regards the dynamic range of the signal processed, and rules out processing between images.

Furthermore, it is increasingly necessary to associate echographic images with other types of images enabling a more complete and more reliable diagnosis to be made. One then speaks of merging clinical examinations. Thus, for example, in the case of lithotrity, it is necessary to have charting by ultrasound imaging of the calculus to be destroyed. It is not then necessary to implement a complete echographic apparatus.

Furthermore, for numerous applications, raw data in the form of values of amplitude and of phase, that is to say before detection, must be available in order to be able to merge them with other information. In order to be able to access these raw data, an attempt has already been made to dismantle an ultrasound imaging apparatus and to install signal taps on the circuits delivering these data, but the results have always been disappointing due to the existence of numerous hardware and software optimizations within an apparatus, so that by “opening the loop”, the operation thereof is disturbed. To avoid these problems, expensive and specific measurement rigs have been constructed in order to access the sought-after raw data.

An object of the present invention is an ultrasound imaging “pack” which is as modular and upgradable as possible, which allows echography to be easily associated with other types of examination, doing so at least expense and without impairing the quality of these examinations.

The ultrasound imaging pack in accordance with the invention is composed of a hardware part for ultrasound imaging preprocessing and of a software part intended to be installed on a microcomputer, the hardware part including at least one ultrasound probe connected to a module of electronic circuits at least part of which is configurable, this module comprising analog FEC circuits, analog/digital converters, an array of logic electronic gates and a high throughput link between this module and the microcomputer, the software part being stored on a removable medium.

In one embodiment, the array of electronic gates is software configurable into specialized chips for channel formation, into a distributed memory, into digital filters, demodulator and control interface.

In another embodiment, the software part configures the configurable elements of the hardware part and installs, on the microcomputer, programs for calculating and presenting ultrasound imaging data.

In another embodiment, the programs installed on the microcomputer comprise programs for producing ultrasound images as gray levels and/or color Doppler images, and/or for producing continuous Doppler information and/or for displaying buttons for controlling processing parameters and/or processing for merging with external data.

In another embodiment, the analog/digital converter is of the software configurable sigma-delta type.

In another embodiment, the array of electronic gates comprises circuits of Field Programmable Gate Arrays type.

In another embodiment, the high throughput link transmits several digital channels.

In another embodiment, the high throughput link is of IEEE1394 type.

In another embodiment, the hardware and software assembly is incorporated into a medical instrumentation assembly.

In another embodiment, the instrumentation assembly comprises equipment for surgery or for microsurgery.

In another embodiment, the medical instrumentation assembly comprises apparatus providing medical data complementary to the ultrasound data.

In another embodiment, the complementary medical data comprise at least one of the following data kinds: data from optical cameras, diagnostic and gynecological and/or cardiac monitoring data.

The present invention will be better understood on reading the detailed description of an embodiment, taken by way of nonlimiting example and illustrated by the appended drawing, in which:

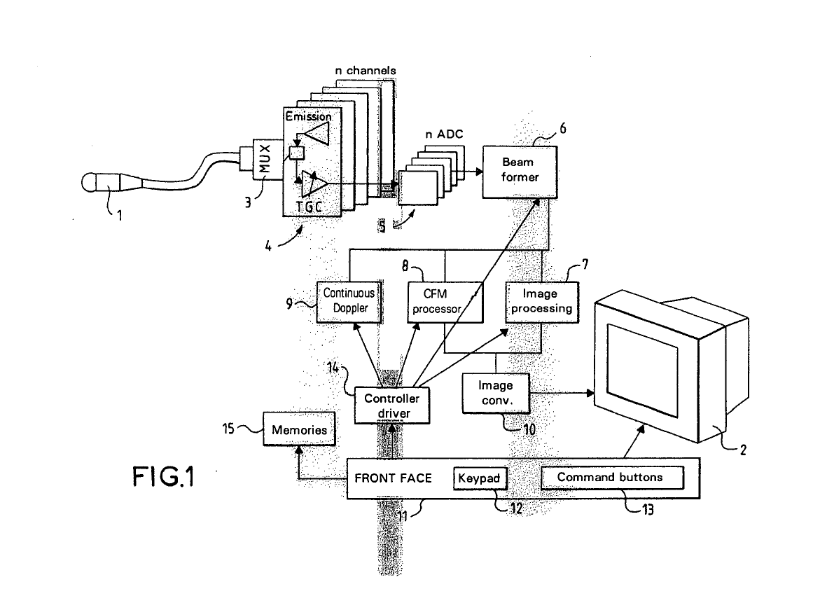

FIG. 1 is a block diagram of an ultrasound system of the prior art;

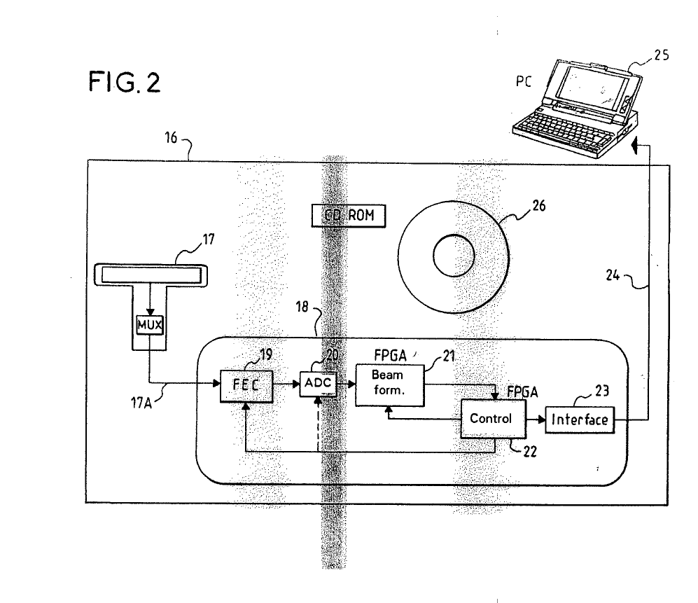

FIG. 2 is a simplified block diagram of an ultrasound system in accordance with the invention; and

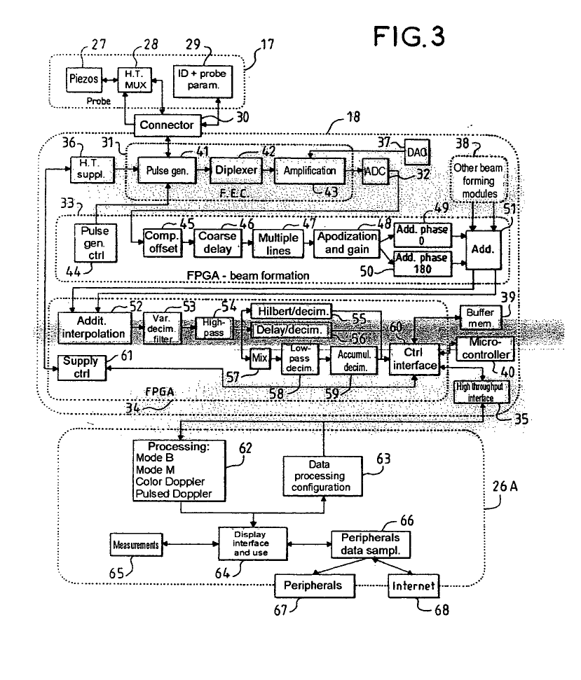

FIG. 3 is a detailed block diagram of an exemplary embodiment of an ultrasound system of FIG. 2.

The ultrasound system of the prior art shown diagrammatically in FIG. 1 comprises a multielement probe 1 which is an acoustic antenna placed in contact with the body to be observed. There are several types of such antennas, that are generally classed into three categories: mechanical antennas, antennas referred to as “phased arrays” and linear-scanning antennas referred to as “linear antennas”. The last two categories are said to be “electronic”. For these two categories, the signals received from a point of the body by the various elementary sensors undergo particular processing compensating for their propagation losses and their delays. These delays are calculated so that all the ultrasound emissions emanating from each of the elementary sensors arrive in phase at the body explored by the probe, and that all the signals reflected by this body are added together in phase.

In the case of a so-called “phased array” antenna, a delay law varying linearly along the antenna, which is plane, is compounded with the parabolic law, this making it possible to ensure the convergence of the ultrasound beam emitted by the probe off its axis, and to gather the echoes from the points situated along an axis corresponding to the delay between each elementary sensor. The series of echoes gathered over time in a given direction is called a line or a channel. The ultrasound image, that is displayed on a display screen 2, consists of the whole set of lines covering the surface of the body to be observed.

The probe 1 is followed by a multiplexer 3, itself followed by an assembly 4 of circuits that is called the “front end conditioning” (FEC). This assembly 4 is responsible for transmitting the ultrasound observation signals to the probe 1 and for gathering the echoes perceived by the probe 1 while amplifying them with the aid of low noise amplifiers, with an amplification level which varies with time so as to compensate for the propagation losses of the ultrasound signal. This assembly 4 also comprises the circuit for separating the transmit signals, which are of high level, from the receive signals (echoes) which are of low level.

The FEC 4 is followed by an assembly 5 of analog/digital converters comprising at most as many converters as there are analog channels (that is to say as many as there are elementary sensors in the probe 1). The digital signals emanating from the various converters of the assembly 5 are processed (filtered, delayed and added together) by a circuit 6 called the “beam former” or “channel former”, this circuit 6 constituting a preprocessor of the echoes. A signal which, as a function of time, corresponds to an image line is gathered at the output of the circuit 6. A total ultrasound image is constructed by repeating the emission/reception operation on the whole set of lines to be explored (moving the probe parallel to the image line).

It will be noted that the parameter n of the number of signals added together simultaneously (and less than or equal to the number of elementary sensors of the probe 1) during beam formation is a very important parameter of an ultrasound imaging apparatus. The quality of the image observed, and in particular its contrast, is directly related to this parameter n. Specifically, if one considers that an echo originating from a point of the body to which the probe is applied gives a signal of amplitude A on one of the elementary sensors of the probe, the amplitude of the signal at the output of the channel formation circuit is n.A for this echo alone. This signal will be n times stronger than the signals that do not arrive at the same time as it.

The beam former 6 is followed by several specialized processors, namely a black and white imaging processor 7, a processor for detecting blood turbulence (so-called “color flow mapping”) 8 and a Doppler processor 9. The processor 7 produces the traditional echographic image based on gray levels, in which the intensity of each point of the image is dependent on the amplitude of the corresponding echo. The processor 7 detects this amplitude, performs various processing operations to avoid speckle due to interference between multiple echoes and performs a dynamic range compression to render the signal suitable for display on the screen 2 whose dynamic range is limited.

The Doppler imaging processor 8 calculates, for each of the points of the image, a value of the mean of the variation in frequency of the echo, corresponding to a mean velocity of the blood. The Doppler images are generally superimposed on the black and white image in color coded form. One then speaks of color images.

The processor 9 provides the complete spectrum of the variations in Doppler frequency, thereby giving an indication as to the turbulent or nonturbulent nature of the blood flow.

The three processors 7 to 9 are connected to an image converter (“scan converter”) 10, which transforms the signal gathered arriving along lines forming a sector into a video image observable on a screen.

The known ultrasound imaging system also comprises a control panel 11 furnished with a keypad 12 and with command buttons 13, to control the monitor 2 and allowing the user to choose and to modify the parameters of the machine, via a command and drive circuit 14 which controls the processors 6 to 9. Furthermore, the ultrasound system comprises various peripherals such as memories 15, and an image recorder (video recorder, DVD recorder, etc.), a reprographic device for printing images faithful to those displayed on the screen of the monitor 2, etc. (not represented in FIG. 1).

Represented in FIG. 2 are the main elements of the hardware and software pack 16 in accordance with the invention. The hardware part comprises an electronic probe 17, with 128 channels for example, advantageously furnished with a demultiplexer, for example, a 128 Π64 demultiplexer (with 64 output channels for the present example of 128 incoming channels) connected by a cable 17A to a module 18 of customizable circuits, described in greater detail with reference to FIG. 3. This module 18 essentially comprises analog FEC circuits 19, an assembly of n analog/digital converters 20, a beam forming device 21, a control device 22 and a high throughput output interface 23 (for example of IEEE 1394 type). The device 22 controls the devices 19, 20, 21 and 23. The interface 23 is connected by a high throughput link 24 to an appropriate microcomputer 25, which may for example be a portable PC and whose hardware part has undergone no modification. The link 24 transmits several digital channels from the module 18 to the PC 25. The assembly 16 also comprises a software part, which is for example stored on a CD-ROM 26, or on any other removable memory medium (DVD-ROM, removable hard disk, etc.) and an installation and usage guide (with, as appropriate, a guide for tests and maintenance) which can be printed, or, preferably, stored on the removable medium 26.

The software part can easily be installed on the PC 25, whose characteristics (sufficient memory and fast processor) make it possible to utilize the software in an optimal manner. The installation of the pack of the invention consists in setting up the electrical connections between the components (between the elements 17, 18 and the PC 25) and in running the software when the CD-ROM 26 has been inserted into the corresponding reader of the PC. This software controls the configuration of the various configurable circuits of the module 18 as a function of the probe used, as a function of the processing to be performed, as well as the configuration of the PC so that the latter can receive the channels formed by the beam former 21, filter them and detect them, convert them into video images, display them on its display screen, do the Doppler calculations, generate the Doppler images, and perform all the other necessary processing.

The user thus has access to all the aspects of the ultrasound imaging data that he will utilize according to his own requirements: conventional ultrasound imaging by applying the chosen probe to the body to be visualized or recovery of raw data originating from the probe, or of partially processed data, with a view to merging them with other types of data.

The application program of the software part comprises not only the operating parameters of the module 18 (waveforms of the signals traveling through the various circuits of this block, excitation voltages for energizing the elementary sensors of the probe, filtering parameters, etc.) but also and especially the actual structure of the processing of the data, and hence the actual structure of the preprocessor formed by the module 18. By changing the user program (written onto the removable medium 26), it is thus possible to considerably change the nature of the data processing performed by the pack of the invention.

The pack of the invention thus has the advantage of low development cost, and especially low manufacturing cost, and this for high performance. The hardware development is limited to the card supporting the circuits of the module 18. This pack is plugged into a traditional probe, but can also use specific probes. The PC 25 is of a type commonly available (for example a 700 MHz processor, a RAM memory with a capacity of 128 Mbits, a CR-ROM reader, and a 10 Gbyte hard disk).

The PC can be optimized simply by loading the appropriate software of the removable medium 26, and as soon as new processing algorithms appear, they can be loaded into the PC without having to physically modify the module 18.

Recent progress in echography pertains especially to the software part of echographs. One may for example cite:
3D imaging which consists in forming 2D contour images, and in constructing a 3D image by moving the probe;
Doppler intensity images;
images of blood velocities parallel to the line of sensors of the probe (where the Doppler velocity is zero);
imaging of deformation or of correlation between various images under a deformation imposed from outside, and calculation of the deformation of the tissues examined;
elasticity imaging by utilizing algorithms of the aforesaid imaging procedures.

All these advanced features can be implemented in the installed pool of products in accordance with the invention simply by updating their software.

By virtue of its configurability, the pack of the invention can be utilized in very varied configurations and applications, thus in fact allowing standardization of its hardware part. To develop new applications, it will not usually be necessary to develop new hardware, but simply new software. New algorithms, if any, for antenna processing (for processing the signals from the probe) may also be installed by software update, for example so as to be able to use adaptive antennas.

Given that the components of microcomputers are being rapidly upgraded, and in particular the capacity of their hard disks (hard disks of more than 100 Gbytes are currently commonly on offer), they enable not just a few images, but an entire lengthy examination to be stored easily. Furthermore, because the information transmitted to the PC is in the form of an amplitude and of a phase of a signal, the latter can be stored and processed several years later with the aid of algorithms not invented at the time of the examination.

Information provided by different sensors is utilized in numerous medical applications. Such is the case, for example, for computer-aided surgery or microsurgery. Optical cameras are used to produce synthetic images that are compared with the real images, provided by other optical cameras, so that the part of the organ to be treated can be better charted. While the optical camera enables only the exterior of this organ to be seen, the acoustic “camera” (the ultrasound probe) makes it possible to “see” inside this organ without opening it, and becomes indispensable to the optimization of the track of the scalpel, thus making it possible to reduce operative trauma. Thus, the pack of the invention is advantageously incorporated into a medical instrumentation assembly, which comprises surgical or microsurgical equipment. This medical instrumentation assembly comprises apparatus providing medical data complementary to the ultrasound data. These complementary medical data are for example data from optical cameras and/or diagnostic and gynecological and/or cardiac monitoring data.

Represented in FIG. 3, is an exemplary embodiment of the pack 16. This pack essentially comprises three parts, namely the probe 17, the module 18 and the software part 26A installed in the PC.

The probe 17 comprises an assembly 27 of piezoelectric sensors, a multiplexer 28 and a memory 29 in which are stored the characteristic parameters of the probe and its identity.

The probe 17 is connected by a cable comprising a connector 30 to the module 18 which comprises five main subassemblies: FEC circuits 31, an analog/digital converter 32, beam former circuits 33, distributed-memory, control, demodulation and interface circuits 34, and an interface 35 for linking to the PC. The circuits 31 to 35 correspond respectively to the elements 19 to 23 of FIG. 2. The module 18 furthermore comprises a high voltage power supply 36 powering the circuits 31, digital/analog converters 37 controlling the amplifiers of the subassembly 31, additional beam forming modules 38 identical to the module 33, which are also connected to the output of the assembly of converters 32, a buffer memory 39 and a microcontroller 40 that are connected to the subassembly 34.

The FEC subassembly 31 comprises an ultrasound pulse generator 41 powered by the power supply 36 and connected on the one hand by the connector 30 to the probe 17, and on the other hand by a diplexer 42 to amplifying circuits 43. These amplifying circuits are controlled by the program of the PC (through a link that is not represented) via the converters 37.

The beam former 33 comprises on the one hand a circuit 44 for controlling the pulse generator 41, and on the other hand, the signal digital processing chain connected to the output of the converter 32 and composed successively of an offset compensator 45, of a coarse delay circuit 46, of a circuit 47 for processing multiple scan lines, of an apodization and amplification circuit 48, of two circuits in parallel 49, 50 for zero phase addition (no phase shift) and for 180° phase shift, respectively, and of a cascaded addition circuit 51. The circuit 51 is connected on the other hand to the output of the circuits 38.

The subassembly 34 comprises, in the order of progression of the signals that it processes: an interpolation and addition circuit 52 connected to the output of the circuit 51, a variable-characteristics and decimation filter 53, a high-pass filter 54 whose output is connected at one and the same time to three circuits: a Hilbert transform and decimation filter 55, a delay and decimation circuit 56, and a mixer 57 followed by a decimation low-pass filter 58 and by a decimator accumulator 59. The outputs of the circuits 55, 56 and 59 are connected to an interface and control circuit 60. The circuit 60 is connected in a bidirectional manner to the memory 39, to the microcontroller 40, to the high throughput interface 35 and to a circuit 61 for controlling and monitoring the power supply 36. The functions undertaken by the various blocks of the subassemblies of the modules 17 and 18 are known per se and will not be described in detail here.

Also represented in FIG. 3, in the form of function blocks, is the software part 26A of the pack of the invention when it is installed on the PC 25 and operational. The software part configures the configurable elements of the hardware part and installs, on the microcomputer 25, programs for calculating and presenting ultrasound imaging data. At input, this software part 26A comprises a function 62 for Doppler color and pulsed Doppler processing in mode B, in mode M, and a function 63 for configuring the configurable circuits of the module 18. These two functions 62 and 63 communicate with the module 18 via the interface 35. The function 62 exchanges data in both directions with the module 18, while the function 63 dispatches configuration orders and the corresponding data to the module 18.

The function 62 controls a display and user interface function 64. This function 64 produces the display on the screen of the PC of the images processed by the function 62, and moreover, transmits the raw or processed data originating from the module 18 to a measurement function 65 and to a function 66 for controlling peripheral hardware 67 and for exchanging data transmitted by an interface 68, which may, for example, be an Internet type interface.

According to a preferred embodiment of the invention, the subassemblies 33 and 34 consist of programmable FPGA (Field Programmable Gate Array) circuits consisting of logic gates, for example of circuits from the Xilinx company. For the subassemblies 33 and 34, the module 18 comprises five such gates, that are configured so as to make them process 4 channels of n=64 sensors or else 2 channels of 128 sensors, or else again, one channel of 128 sensors. The assembly of circuits 31 to 34, embodied according to current technologies, occupies a space 20×30 cm when the FECs 31 are not integrated. If specific analog integrated circuits were utilized to embody these FECs, the space occupied by the elements 31 to 34 could be divided by 3.

According to a variant embodiment, input multiplexers that make it possible to use either probes of the “phased array” type with 64 sensors or with 128 sensors or linear or curved probes with 128, 192 or 256 sensors, are added just ahead of the FEC 31.

According to an advantageous embodiment, the analog/digital converters 32 are not complete: use is made of ultra-fast converters, but of small “depth” (that is to say at the minimum a data definition on 1 input bit and 1 sign bit). By way of example, the maximum frequencies of the echographic signals are about 15 MHz. Compliance with the Nyquist criterion leads to the use of converters 32 whose clock frequency is 33 MHz. The minimum depth of the information from the sensors is 10 bits and 1 sign bit, and the embodiment described here uses said ultra-fast converters, operating at clock frequencies of a few hundred MHz. A calculation algorithm referred to as “sigma-delta” makes it possible to utilize oversampling to calculate the missing depth bits. This algorithm can be stored in the FGPA gate array of the module 18, in software form, that is to say through orders originating from the PC and passing through the interface 35. Thus, the configurability of the pack of the invention is extended to the analog/digital converter.

According to another embodiment, the converter 32 is embodied with the aid of circuits with a clock frequency of about 300 MHz, but a few bits in depth (for example 8 bits). By calculation, these 8-bit converters are transformed into 11-bit converters with a clock frequency of about 33 MHz.

In these embodiments with low-depth converters, the advantage lies in the great reduction in the number of outputs of the converter that have to be connected to the inputs of the subassembly 33, thus making it possible to very substantially decrease the space occupied by the module 18.

According to yet another embodiment; the probe comprises several rows of sensors. It is said to be of the 1.5 D type. Of course, to be able to plug in such a probe, the module 18 must be equipped with an appropriate specific connector. Images with elevational channel forming can be obtained by loading corresponding operating software into the module 18.

According to yet another embodiment, a two-dimensional probe is used, that is to say one which comprises a 2D ultrasound sensor array. Of course, in this case, the space occupied by the FECs is increased, this being dependent on the total number of elementary sensors of the probe. But on the other hand, the beam forming function comprises only a few complementary circuits.

1. An ultrasound imaging hardware and software pack comprising:
a hardware part for ultrasound imaging preprocessing and of a software part intended to be installed on a microcomputer, the hardware part including at least one ultrasound probe connected to a module of electronic circuits at least part of which is configurable, this module comprising analog FEC circuits, an assembly of analog/digital converters, an array of logic electronic gates and a high throughput link between this module and the microcomputer, the software part being stored on a removable medium.

2. The pack as claimed in claim 1, wherein the array of electronic gates is software configurable into specialized chips for channel formation, into a distributed memory, into digital filters, demodulator and control interface.

3. The pack as claimed in claim 1, wherein the software part configures the configurable elements of the hardware part and installs, on the microcomputer, programs for calculating and presenting ultrasound imaging data.

4. The pack as claimed in claim 3, wherein the programs installed on the microcomputer comprise programs for producing ultrasound images as gray levels and/or color Doppler images, and/or for producing continuous Doppler information and/or for displaying buttons for controlling processing parameters and/or processing for merging with external data.

5. The pack as claimed in claim 1, wherein the analog/digital converter is of the software configurable sigma-delta type.

6. The pack as claimed in claim 2, wherein the array of electronic gates comprises circuits of Field Programmable Gate Arrays (FPGA) type.

7. The pack as claimed in one of the preceding claim 1, wherein the high throughput link transmits several digital channels.

8. The pack as claimed in claim 1, wherein the high throughput link is of IEEE1394 type.

9. The pack as claimed in claim 1, wherein it is incorporated into a medical instrumentation assembly.

10. The pack as claimed in claim 9, wherein the instrumentation assembly comprises equipment for surgery or for microsurgery.

11. The pack as claimed in claim 9, wherein the medical instrumentation assembly comprises apparatus providing medical data complementary to the ultrasound data.

12. The pack as claimed in claim 11, wherein the complementary medical data comprise at least one of the following data kinds: data from optical cameras, diagnostic and gynecological and/or cardiac monitoring data.

13. The pack as claimed in claim 2, wherein the software part configures the configurable elements of the hardware part and installs, on the microcomputer, programs for calculating and presenting ultrasound imaging data.

14. The pack as claimed in claim 2, wherein the analog/digital converter is of the software configurable sigma-delta type.

15. The pack as claimed in claim 3, wherein the array of electronic gates comprises circuits of Field Programmable Gate Arrays (FPGA) type.

16. The pack as claimed in claim 3, wherein the analog/digital converter is of the software configurable sigma-delta type.

17. The pack as claimed in claim 4, wherein the array of electronic gates comprises circuits of Field Programmable Gate Arrays (FPGA) type.

18. The pack as claimed in claim 5, wherein the array of electronic gates comprises circuits of Field Programmable Gate Arrays (FPGA) type.

19. The pack as claimed in claim 7, wherein the high throughput link is of IEEE1394 type.

20. The pack as claimed in claim 10, wherein the medical instrumentation assembly comprises apparatus providing medical data complementary to the ultrasound data.
